(19) United States
(12) Patent Application Publication
(10) Pub. No.: US 2024/0216551 A1
(43) Pub. Date: Jul. 4, 2024

(54) RADIOPHARMACEUTICALS BASED ON ((R)-1-((6-HYDRAZINYLNICOTINOYL)-D-ALANYL)PYRROLIDIN-2-YL)BORONIC ACID (HYNIC-IFAP) FOR DETECTING THE OVEREXPRESSION OF FIBROBLAST ACTIVATION PROTEIN

(71) Applicants: Guillermina FERRO-FLORES, LA MARQUESA, OCOYOACAC (MX); Blanca Elí OCAMPO-GARCÍA, LA MARQUESA, OCOYOACAC (MX); INSTITUTO NACIONAL DE INVESTIGACIONES NUCLEARES, LA MARQUESA OCOYOACAC (MX)

(72) Inventors: Guillermina FERRO-FLORES, LA MARQUESA OCOYOACAC (MX); Blanca Elí OCAMPO-GARCÍA, LA MARQUESA OCOYOACAC (MX); Myrna Alejandra LUNA-GUTIÉRREZ, LA MARQUESA OCOYOACAC (BE); Clara Leticia SANTOS-CUEVAS, LA MARQUESA OCOYOACAC (MX); Nallely Patricia JIMÉNEZ-MANCILLA, LA MARQUESA OCOYOACAC (MX); Erika Patricia AZORIN-VEGA, LA MARQUESA OCOYOACAC (MX)

(73) Assignee: INSTITUTO NACIONAL DE INVESTIGACIONES NUCLEARES, LA MARQUESA OCOYOACAC (MX)

(21) Appl. No.: 18/288,838

(22) PCT Filed: Oct. 14, 2021
(86) PCT No.: PCT/MX2021/050055

§ 371 Date: Oct. 30, 2023

(30) Foreign Application Priority Data
Apr. 30, 2021 (MX) MX/A/2021/005089

(51) Int. Cl. A61K 51/04

(52) U.S. Cl. CPC A61K 51/0455; A61K 51/0497; A61K 2123/00

(57) ABSTRACT

This invention relates to new fibroblast activation protein (iFAP) inhibitory radiopharmaceuticals based on the molecule ((R)-1-((6-hydrazinylnicotinoyl)-D-alanyl)pyrrolidin-2-yl)boronic acid (HYNIC-iFAP), where the hydrazine nitrogens of HYNIC act as favorable chemical groups for the interaction of the HYNIC-iFAP molecule with phenylalanine (Phe-350 and Phe-351), glutamic acid (Glu-203 and Glu-204) and with serine (Ser-624) in the active center of fibroblast activation protein (FAP), coupled with the conventional use of HYNIC as a chelating agent for the radiometal ^{99m}Tc, where ethylenediamine diacetic acid (EDDA) is used to complete the coordination sphere of the radiometal. The new radiopharmaceutical from ^{99m}Tc-EDDA/HYNIC-iFAP (^{99m}Tc-HYNIC-iFAP) detects, with high affinity in vivo, FAP expressed in the microenvironment of malignant tumors of epithelial origin using nuclear medicine SPECT molecular imaging techniques. The object of this invention is to provide a new SPECT-specific radiopharmaceutical with high sensitivity for the detection of FAP protein expression in the tumor microenvironment, based on boronPro-type inhibitors (molecular target radiopharmaceutical).

### CROSS-REFERENCE TO RELATED APPLICATIONS

This application is a U.S. national stage entry under 35 U.S.C. § 371 of PCT International Patent Application No. PCT/MX2021/050055, filed Oct. 14, 2021, which claims priority to Mexican Patent Application No. MX/a/2021/005089, filed Apr. 30, 2021, the contents of each of which are incorporated herein by reference in their entirety.

### TECHNICAL FIELD OF THE INVENTION

The present invention relates to new radiopharmaceuticals based on the fibroblast activation protein (iFAP) inhibitor ((R)-1-((6-hydrazinylnicotinoyl)-D-alanyl)pyrrolidin-2-yl)boronic acid (HYNIC-iFAP), where the hydrazine nitrogens of HYNIC act as favorable chemical groups for the interaction of the HYNIC-iFAP molecule with phenylalanine (Phe-350 and Phe-351), glutamic acid (Glu-203 and Glu-204) and serine (Ser-624), the essential active center of fibroblast activation protein (FAP), coupled with the conventional use of HYNIC as a chelating agent for the radiometal ^{99m}Tc. In particular, the new radiopharmaceutical HYNIC-iFAP labeled with ^{99m}Tc, detects with high affinity in vivo, the FAP expressed in the tumor microenvironment by SPECT molecular imaging techniques in nuclear medicine.

### Background

Fibroblast activation protein (FAP) is a type II serine protease that cleaves peptides located downstream of proline residues with dipeptidyl-peptidase and endopeptidase activities. FAP is highly expressed on the cell surface of the activated stromal fibroblast present in most human epithelial tumors, but not in normal fibroblasts. Cancer-associated fibroblasts contribute up to 90% of the macroscopic tumor mass [Hamson et al. Understanding fibroblast activation protein (FAP): substrates, activities, expression and targeting for cancer therapy. Proteomics Clin. Appl., 2014, 8, 454-463].

Specific FAP inhibitors were first developed as potential anticancer drugs [Aertgeerts et al. Structural and kinetic analysis of the substrate specificity of human fibroblast activation protein α. J. Biol. Chem., 2005, 280, 19441-19444; Edosada et al. Selective inhibition of fibroblast activation protein protease based on dipeptide substrate specificity. J. Biol. Chem. 2006, 281, 7437-7444; Tran et al. Synthesis and structure-activity relationship of N-acyl-Gly-, N-acyl-Sar-and N-blocked-boroPro inhibitors of FAP, DPP4, and POP. Bioorg. Med. Chem. Lett., 17, 2007, 1438-1442]. Of these, the most relevant are two groups of highly selective compounds, one based on a quinoline-cyanopyrrolidin structure and the other based on pyrrolidin-boronic acid or also known as proline-boronic acid (boronPro). Regarding the first group, Jansen et al. initially reported the synthesis of 39 new FAP inhibitors to explore the structure-activity relationship of the 4-quinolinoyl-Glycyanopyrrolidin scaffold [Jansen et al. Extended structure activity relationship and pharmacokinetic investigation of (4-quinolinoyl)-glycyl-2-cyanopyrrolidin inhibitors of fibroblast activation protein J. Med. Chem., 2014, 57, 3053-3074]. The authors reported that FAP imposes stringent structural requirements in order to form covalent bonds between the enzyme and the nitrile group present in these molecules [Jansen et al. Selective inhibitors of fibroblast activation protein (FAP) with a (4-quinolinoyl)-glycyl-2-cyanopyrrolidin scaffold. ACS Med. Chem. Lett., 2013, 4, 491-496]. They also found that N-pyridines give rise to FAP inhibitors of high selectivity towards other post-proline cleavage enzymes, such as dipeptidyl-peptidases (DPP) and prolyl-oligopeptidase (PREP). Although the quinolinoyl fragment confers the molecule a higher affinity for FAP, when this residue was replaced by another azaheteroaromatic substituent, the affinity for FAP decreased drastically. Furthermore, the addition of fluorine or difluoro to the cyanopyrrolidin ring fragment was found to improve the affinity and selectivity for FAP. When the authors replaced the glycine residue with some other amino acids, the affinity for PREP increased and the potency of FAP decreased significantly. As a result of this study, the authors identified the N-(4-quinolinoyl)-glycyl-(2-cyanopyrrolidin) scaffold as the best inhibitor of FAP. Pharmacokinetic studies in mice of the selected FAP inhibitor showed high bioavailability after oral administration, with a short plasma half-life and long-lasting in vivo FAP inhibition.

At the same time, Poplawsky et al. designed and characterized more than 20 proline-boronic-based inhibitors for FAP and PREP [Poplawski et al. Identification of selective and potent inhibitors of fibroblast activation protein and prolyl oligopeptidase. J. Med. Chem. 2013, 56, 3467-3477]. The authors reported that the affinity for FAP could be increased by using a protonated pyridinic nitrogen atom, because it forms a hydrogen bond with the carbonyl oxygen of glutamic acid (Glu-204) present in FAP, but absent in PREP. The authors also found that the endopeptidase activity of FAP has extremely stringent requirements, accepting only small amino acids such as glycine or D-alanine. Although D-alanine is preferred because the inhibitor retains FAP inhibitory potency in the nanomolar range and provides 360-fold selectivity for FAP over PREP. Based on these results, Poplawski et al. established that N-(pyridin-4-carbonyl)-D-Ala-boroPro was the best fibroblast activation protein inhibitor found.

Despite the large number of synthesized and characterized FAP inhibitors, only a limited number of them have been radiolabeled for medical purposes. In 2015, the first radiolabeled boronic acid-based FAP inhibitor with iodine-125 (¹²I-MIP-1232) was reported for atherosclerotic plaque imaging, although it is important to clarify that I-125 is not a useful radionuclide for single photon emission computed tomography (SPECT) or positron emission tomography (PET) imaging [Meletta et al. Evaluation of the radiolabeled boronic acid based FAP inhibitor MIP-1232 for atherosclerotic plaque imaging. Molecules, 2015, 20, 2081-2099].

In 2018, Lindner and Loktev reported the conjugation of 2,2′,2″,2′″-(1,4,7,10-tetraazacyclododecane-1,4,7,10-tetrayl)tetraacetic acid (DOTA) to the 4-quinolinoyl-Gly-cyanopyrrolidin scaffold in order to label it with suitable radionuclides for diagnostic imaging or therapeutic purposes [Lindner et al. Development of quinoline-based theranostic ligands for the targeting of fibroblast activation protein. J. Nucl. Med., 2018, 59, 1415-1422; Loktev et al. A tumor-imaging method targeting cancer-associated fibroblasts. J. Nucl. Med., 2018, 59, 1423-1429]. The authors developed fifteen derivatives using different positions to conjugate DOTA to the 4-quinolinoyl-Gly-cyanopyrrolidin scaffold in order to improve tumor uptake and retention time of FAP inhibitor (FAPI) derivatives. The 15 different FAPIs were radiolabeled with ¹⁷⁷Lu, ⁹⁰Y or ⁶⁸Ga. Of all the radiolabeled and reported FAPI complexes, ⁶⁸Ga-FAPI-02 and ⁶⁸Ga-FAPI-04 proved to be the most suitable agents for diagnostic applications. Subsequently, the same authors reported other derivatives of the same framework numbered consecutively from FAPI-21 to FAPI-55 [Loktev et al. Development of Fibroblast Activation Protein-Targeted Radiotracers with Improved Tumor Retention. J. Nucl. Med., 2019, 60, 1421-1429]. Substitution of DOTA with 1,4,7-triazacyclononane-N, N′,N″-triacetic acid (NOTA) gave rise to the derivative FAPI-74, which can be labeled with ⁶⁸Ga and with ¹⁸F (¹⁸F-AIF, in the presence of AlCl₃) [Giesel et al. FAPI-74 PET/CT Using Either ¹⁸F-AIF or Cold-Kit ⁶⁸Ga Labeling: Biodistribution, Radiation Dosimetry, and Tumor Delineation in Lung Cancer Patients. J. Nucl. Med., 2021, 62, 201-207]. In particular, FAPI-35 was developed for labeling with ^{99m}Tc and possibly ¹⁸⁸Re [Lindner et al. Design and Development of ^{99m}Tc-Labeled FAPI Tracers for SPECT Imaging and ¹⁸⁸Re Therapy. J. Nucl. Med., 2020, 61, 1507-1513].

The first FAP inhibitors used in humans for PET scans were ⁶⁸Ga-FAPI-02 and ⁶⁸Ga-FAPI-04 [Giesel et al. ⁶⁸Ga-FAPI PET/CT: biodistribution and preliminary dosimetry estimate of 2 DOTA-containing FAP-targeting agents in patients with various cancers. J. Nucl. Med., 2019, 60, 386-392; Kratochwil et al. ⁶⁸Ga-FAPI PET/CT: Tracer Uptake in 28 Different Kinds of Cancer. J. Nucl. Med., 2019, 60, 801-805].

Interestingly, the ⁶⁴Cu-/²²⁵Ac-FAPI-04 theranostic couple, which demonstrated its utility in the treatment of pancreatic cancer overexpressing FAP in murine models. Therapy targeting fibroblast activation protein is effective in cancer treatment and could contribute to the establishment of new treatment strategies [Watabe et al. Theranostics Targeting Fibroblast Activation Protein in the Tumor Stroma: ⁶⁴Cu- and ²²Ac-Labeled FAPI-04 in Pancreatic Cancer Xenograft Mouse Models. J. Nucl. Med., 2020, 61, 563-569].

However, prior to any radiotherapeutic treatment, the uptake of the radiopharmaceutical in the tumors or their metastases must be evaluated by nuclear imaging in order to confirm whether or not the treatment will be useful for the patient, as well as to determine the necessary activity to administer in order to deliver the ablative radiation dose to the tumors, i.e. personalized medicine is practiced. For this, it is necessary to use FAP inhibitor diagnostic radiopharmaceuticals in order to obtain molecular images by PET or SPECT. Of these two techniques, PET is the one with the highest spatial resolution and sensitivity, so that, as described above, the diagnostic FAP inhibitor radiopharmaceuticals so far developed and applied in clinical studies, use ⁶⁸Ga and ¹⁸F bound to 4-quinolinoyl-Gly-cyanopyrrolidin derivatives, which are radiopharmaceuticals for PET, and only one study has used a derivative of ^{99m}Tc also bound to quinoline-cyanopyrrolidin (see table below). Nevertheless, nationally and internationally, SPECT studies represent more than 70% of the total in nuclear medicine due to their lower cost and greater availability of equipment and radionuclides, since it is not necessary to have a cyclotron in or near hospitals. For SPECT imaging, the most commonly used radionuclide is ^{99m}Tc, and there is no publication dedicated to a study on radiopharmaceuticals for FAP imaging based on pyrrolidin-boronic acid derivatives or boron-Pro.

TABLE 1
Chemical structure of radiopharmaceuticals of ⁶⁸Ga, ¹⁸F and ^{99m}Tc FAP inhibitors
so far developed and used in clinical studies by PET and SPECT techniques.

| | NAME |
|---|---|
| CHEMICAL STRUCTURE | AND USE |
| 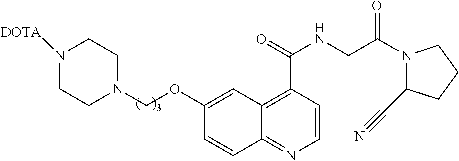 | FAPI-02 Radiolabeled with ⁶⁸Ga PET imaging |
| 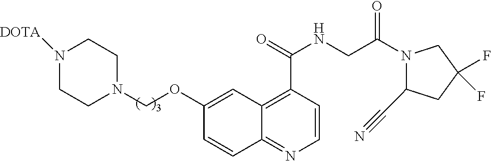 | FAPI-04 Radiolabeled with ⁶⁸Ga PET imaging |
| 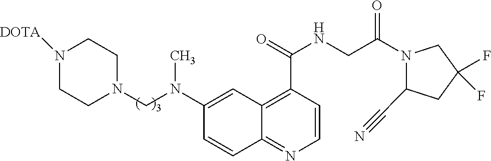 | FAPI-46 Radiolabeled with ⁶⁸Ga PET imaging |
| 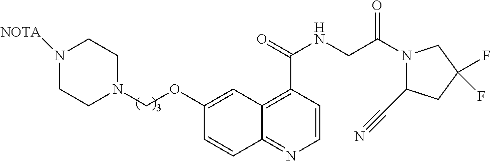 | FAPI-74 Radiolabeled with ⁶⁸Ga and ¹⁸F PET imaging |
| 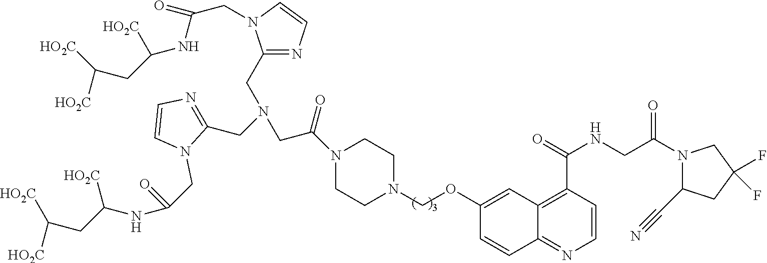 | FAPI-34 Radiolabeled with ^{99m}Tc SPECT imaging |

### DETAILED DESCRIPTION OF THE INVENTION

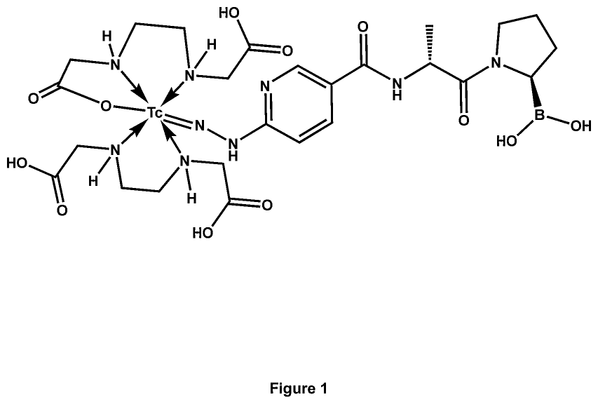

Novel fibroblast activation protein (iFAP) inhibitory radiopharmaceuticals based on the ((R)-1-((6-hydrazinylnicotinoyl)-D-alanyl)pyrrolidin-2-yl)boronic acid (HYNIC-iFAP) molecule are presented for patent purposes, where the hydrazine nitrogens of HYNIC act as favorable chemical groups for the interaction of the HYNIC-IFAP molecule with phenylalanine (Phe-350 and Phe-351), glutamic acid (Glu-203 and Glu-204) and with serine (Ser-624) in the active center of fibroblast activation protein (FAP), coupled with the conventional use of HYNIC as a chelating agent for the radiometal ^{99m}Tc, where ethylenediamine diacetic acid (EDDA) is used to complete the coordination sphere of the radiometal. The new radiopharmaceutical from ^{99m}Tc-EDDA/HYNIC-iFAP (^{99m}Tc-HYNIC-iFAP) detects, with high affinity in vivo, FAP expressed in the microenvironment of malignant tumors of epithelial origin using nuclear medicine SPECT molecular imaging techniques. FIG. 1 shows schematically the structure of the ^{99m}Tc radiopharmaceutical to be patented based on the HYNIC-iFAP structure.

Based on docking study, the HYNIC-IFAP derivative a molecular [((R)-1-((6hydrazinylnicotinoyl-)-Dalanyl)-pyrrolidin-2-yl)boronic] was designed and synthesized, which was compared in affinity and inhibition constant (Ki) with two of the most representative structures of boronPro reported in the literature, N-acyl-Gly-boroPro [Tran et al. Synthesis and structure-activity relationship of N-acyl-Gly-, N-acyl-Sar-and N-blocked-boroPro inhibitors of FAP, DPP4, and POP. Bioorg. Med. Chem. Lett., 17, 2007, 1438-1442] and N-(pyridin-4-carbonyl)-D-Ala-boroPro [Poplawski et al. Identification of selective and potent inhibitors of fibroblast activation protein and prolyl oligopeptidase. J. Med. Chem. 2013, 56, 3467-3477]. The affinity of another derivative of the HYNIC-iFAP molecule, where it was conjugated to S-2-(4-Isothiocyanatobenzyl)-DOTA (DOTA-Bz-NCS-HYNIC-iFAP), was also obtained in order to study whether the affinity of this derivative is suitable for potential labeling with other DOTA-like radiometals, such as ⁶⁸Ga, ¹⁷⁷Lu, ⁶⁴Cu, ²²⁵Ac, etc. For this purpose, all structures were generated in ChemDraw (.cdx format) and the 3D structure was exported in .pdb format via Chem3D software. Using the molecular editor AVOGADRO 1.2.0, the molecular geometry was optimized using a universal force field (UFF), due to the presence of a boron atom in each structure, with a total of 10,000 steps. Subsequently, a second geometry optimization was performed using the semi-empirical Quantum Chemistry software MOPAC2016 with a PM7 level of theory exporting the resulting spatial configuration to .pdb format.

The crystal structure of the alpha subunit of fibroblast activating protein (FAP) was obtained from the RSCB Protein Data Bank database (PDB ID: 1Z68). In order to use it as a receptor macromolecule in molecular docking calculations, the molecule was edited in BIOVIA Discovery Studio 2021 to remove water molecules and residues manifested in X-ray diffraction, leaving only the main amino acid chain in the pdb file. The A chain of the dimer represented in the model was also removed, leaving only the B chain. Because the three-dimensional model of the macromolecule involves a resolution of 2.60 Å, a homology modeling step was performed through the SWISS-MODEL online platform. The resulting structure was saved in pdb format for use as a receptor. Both the receptor and the HYNIC-iFAP-derived structures were prepared with the OPEN BABEL GUI 2006 library indicating the addition of missing hydrogens and their molecular optimization for physiological pH (pH=7.4), again generating the structures in .pdb format. The AutoDock Tools 1.5.6 software package was used to configure the receptor as a macromolecule and each of the ligands in separate files, exporting the files in .pdbqt extension. The search box was configured at the hydrophobic site S1 around the Ser-624 of the receptor as suggested by Poplawsky and co-workers in 2013, as a center the XYZ coordinates 18.948, 10.676, 28.989 respectively and a size of 90 on each axis of the box were set. Regarding the ligands it was necessary to modify the <<AD4_parameters.dat>> file to add the parameters for the Boron atom available at http://mgldev.scripps.edu/pipermail/autodock/2009-March/005439.html.

The execution of the protein-ligand molecular docking was performed with the AutoDock 4.2.6 package, previously calculating the necessary .map grids with the AutoGrid 4.2.6 tool. The log file with the molecular docking results was visualized in AutoDock Tools by exporting to .pdb format the complex with the best affinity score. Visualization and analysis of distances and interactions was performed by BIOVA Discovery Studio Visualizer 2021.

The inhibition constant (Ki) was calculated using the equation:

$$Ki = {e}^{[ \frac{Affinity}{RT} ]}$$

where:
Affinity: Corresponds to the value obtained in the AutoDock 4.2.6 scoring function.
R: Corresponds to the value of the thermodynamic constant of ideal gases (0.00198179 kcal/mol K).
T: Corresponds to the temperature value in absolute scale 298.15 K that AutoDock 4.2.6 handles in the molecular coupling calculations.

The Ki value is obtained in Molar units (M). Table 2 presents the affinity scores, Ki inhibition constants, and distance to Ser624 for each ligand. As evidenced in Table 2, the HYNIC-iFAP molecule showed a 26-fold lower inhibition constant (Ki=0.536 nM) with respect to the N-(pyridin-4-carbonyl)-D-Ala-boroPro derivative (Ki=14.07), which means a higher inhibitory potency of HYNIC-iFAP by FAP.

In agreement with the interaction map of HYNIC-iFAP and the amino acid residues of the FAP active center obtained in the molecular docking study, the increased affinity of HYNIC-IFAP, with respect to N-(pyridin-4-carbonyl)-D-Ala-boroPro, is due to the presence of the hydrazine nitrogens of HYNIC, which favor van der Waals-type interactions and hydrogen bonds of the HYNIC-iFAP molecule in its coupling to the active center of FAP, mainly with residues Glu-203, Glu-204, Phe-350 and Phe-351, as well as a closer proximity for interaction with Ser-624 (interaction map of amino acid residues of FAP with the HYNIC-iFAP ligand, obtained with the molecular docking methodology described above). The high affinity and FAP inhibitory potency of the DOTA-Bz-NCS-HYNIC-IFAP ligand indicates its potential to be used in the preparation of new diagnostic and therapeutic radiopharmaceuticals, as it could be a useful ligand to be labeled with radionuclides capable of being chelated by the DOTA macrocycle, such as ⁶⁸Ga, ⁶⁴Cu, ¹⁷⁷Lu and ²²⁵Ac.

### Method of Preparation of the Radiopharmaceutical of the Invention

For the synthesis of the HYNIC-iFAP molecule, Boc-pyrrolidin was initially dissolved in ethyl ether/TMEDA under a nitrogen atmosphere and at −40° C. It was subsequently reacted with a solution of s-BuLi (in cyclohexane) at 5° C. B(OMe)₃ was then added and the extraction purification process was carried out (first extraction with 2M NaOH, followed by acidification with 2M HCl, final extraction with EtOAc and evaporation of the solvent), obtaining Boc-pyrrolidin-boronic acid, to which pinanediol was added and the R diasteroisomer was obtained by HPLC separation (microporasil column). After Boc deprotection, D-alanine coupling was performed followed by succinimidyl-N-boc-HYNIC coupling using HATU/DIPEA. Finally, the compound was deprotected with TFA, purified by reverse phase HPLC and lyophilized. The final product was ((R)-1-((-6hydrazinylnicotinoyl-)-Dalanyl)pyrrolidin-2-yl)boronic acid (HYNIC-iFAP), which presented the expected mass spectrum: m/z 322 (calcd. 321) [M+H]⁺; m/z 642 (calcd. 321) 2×[M+H]⁺. ¹H-NMR (300 MHZ, DMSO), δ (ppm): 8.5-6.7 (s, 1H, —CH⁶—, arom. pyridine), 9.7 (s, 1H, —NH—NH—), 8.3 (s, 1H, —CH²—NHCO), 3.1-3.3 (m, 1H, CH₂ CHB).

Reversed-phase HPLC analysis of the freeze-dried white solid showed a chemical purity of the compound of 95%.

HYNIC-IFAP (30 μg) was formulated as a lyophilized dosage form containing 10 mg EDDA, 20 mg tricine, 20 μg stannous chloride and 50 mg mannitol. Said formulation, when reconstituted with 1 mL of a solution of 0.2 M, pH 7 and 1 mL of sodium pertechnetate solution (^{99m}TcO₄Na), obtained in situ from a 99Mo/^{99m}Tc generator, yields the compound to be patented ^{99m}Tc-EDDA/HYNIC-IFAP (^{99m}Tc-HYNIC-iFAP) with a radiochemical purity greater than 98% as determined by reverse phase HPLC.

The radiopharmaceutical remains stable with a radiochemical purity greater than 95% after 24 h of labeling. In vitro stability tests in human serum show serum protein binding of 2.1±0.3% and high radiochemical stability (>95%). For the evaluation of the in vitro specificity of ^{99m}Tc HYNIC-IFAP, we used tumor stroma (patient biopsy) from two different molecular subtypes of breast cancer: a luminal B, characterized by high expression of estrogen receptor (ER) and human epidermal growth factor receptor type 2 (HER2), and a triple negative (TNBC) due to null expression levels of ER, progesterone receptor (PR) and HER2. We chose these tumor stroma as in a previous study in different breast tumor phenotypes, TNBC tumors were shown to express the highest levels of FAP while luminal B tumors expressed the lowest levels of FAP [Park et al. Differential expression of cancer-associated fibroblast-related proteins according to molecular subtype and stromal histology in breast cancer Breast. Cancer Res. Treat., 2015, 149, 727-741]. The results showed an uptake of ^{99m}Tc HYNIC-iFAP of 7.8±1.2% of the radioactivity added to the stroma of TNBC tumors, which was significantly higher (P<0.05, t-student) than the radioactivity taken up by the stroma of luminal B tumors (2.3±0.3% of the radioactivity added).

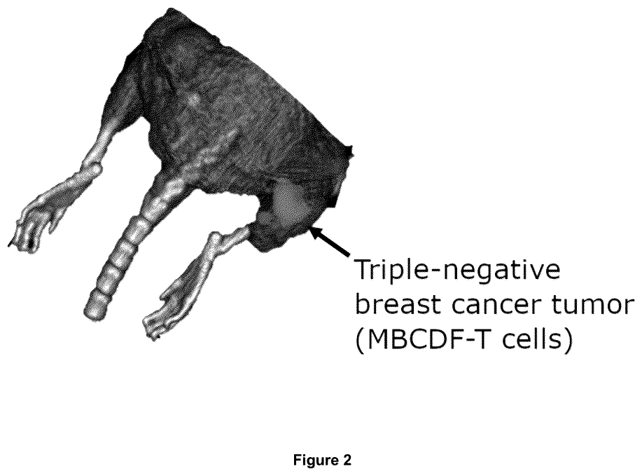

The compound showed no toxicity or adverse effects when administered at a dose of 40 mg/kg to laboratory balb-C mice. Micro-SPECT/CT imaging of ^{99m}Tc-HYNIC-iFAP in athymic mice with induced triple-negative breast cancer tumors (MBCDF-T cells) showed an uptake in tumors of 9.2±1.4% of the administered activity per gram of tissue (% ID/g) (FIG. 2) with rapid elimination mainly via the kidney.

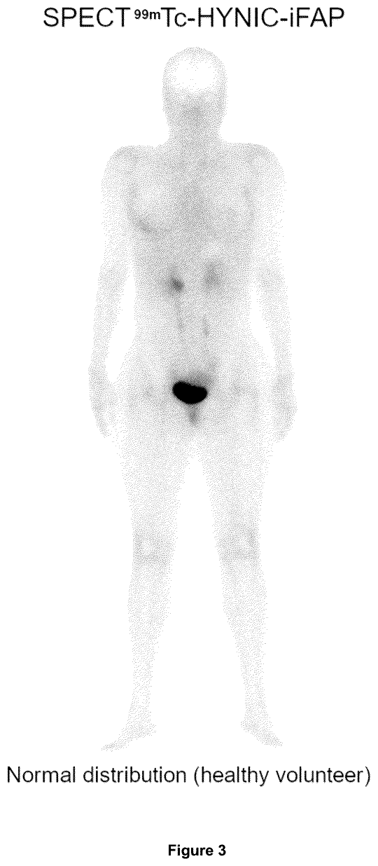

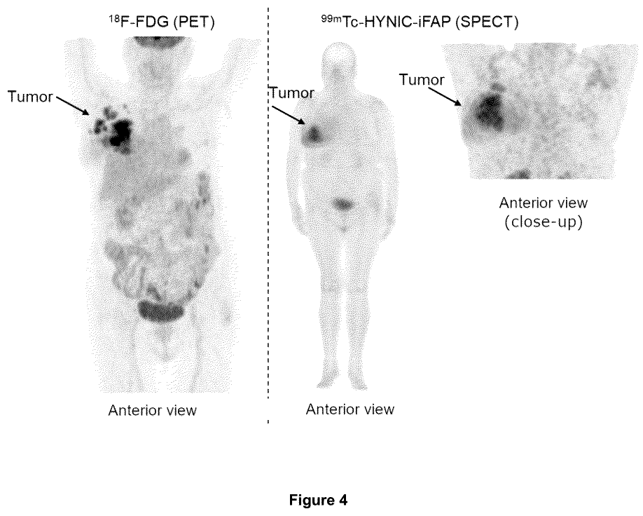

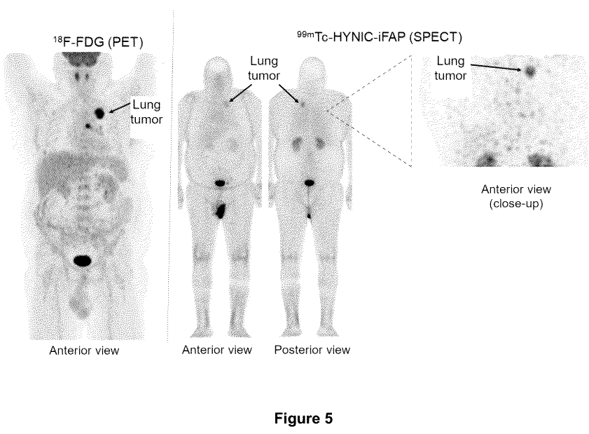

Biokinetic and dosimetry tests in healthy volunteers show rapid blood clearance with increased renal uptake and excretion, and an effective dose of 2.0±0.5 mSv per 740 MBq administered. FIG. 3 shows a SPECT image of the radiopharmaceutical ^{99m}Tc-HYNIC-iFAP obtained in a healthy volunteer at 1 h post-administration. FIG. 4 shows the PET and SPECT image of the same patient with triple negative breast cancer who was administered both ¹⁸F-FDG (PET, control radiopharmaceutical, gold standard for detection of tumor metabolism) and ^{99m}Tc-HYNIC-IFAP (SPECT), showing that both radiopharmaceuticals detect with high sensitivity breast cancer tumors; FIG. 5 shows the PET and SPECT image of the same lung cancer patient (poorly differentiated lung adenocarcinoma with predominantly solid pattern) who was administered both 18F-FDG (PET, control radiopharmaceutical, gold standard for the detection of tumor metabolism) and ^{99m}TC-HYNIC-IFAP (SPECT), showing that both radiopharmaceuticals detect lung tumors with high sensitivity, although in the case of ^{99m}Tc-HYNIC-IFAP, the high uptake is associated with the recognition of FAP expression in the tumor microenvironment. These images confirm and are the main evidence that, due to its FAP activity inhibition properties enhanced by the nitrogens present in HYNIC hydrazine, the ^{99m}Tc-HYNIC-iFAP is able to detect tumor lesions in a specific manner.

In conclusion, the ^{99m}Tc-HYNIC-IFAP is obtained with the following characteristics:
Radiochemical purity greater than 98%.
Ability of the radiopharmaceutical to detect in vivo and specifically to the microenvironment of tumors expressing fibroblast activation protein by nuclear medicine single photon emission computed tomography (SPECT).
In addition to the molecular recognition of the boronPro residue, the radiopharmaceutical to be patented based on ^{99m}Tc, has the ability to significantly capture and detect with high sensitivity the FAP-expressing tumor microenvironment, due to the increased affinity (low Ki value; Ki=0.536) conferred by the presence of the hydrazine nitrogens in the HYNIC molecule, which allows it to interact efficiently in docking with the active site of the FAP enzyme for detection by SPECT imaging.

TABLE 2
Affinity scores determined by molecular docking (AutoDock), as well as the
inhibition constants (Ki), and interaction distance between the boron residue and Serine 624
of fibroblast activation protein (FAP) for each boroPro ligand.

| | | | Distance |
|---|---|---|---|
| | Affinity | Ki | Ser624-B |
| boroPro Ligand | (kcal/mol) | (nM) | (Å) |
| N-acyl-Gly-boroPro 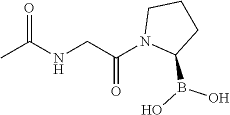 | −4.80 | 296.46 | 3.398 |
| N-(pyridine-4-carbonyl)-D-Ala-boronPro) 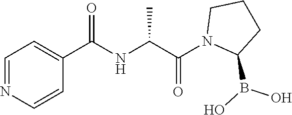 [SMILES: C[C@@H](NC(=O)c1ccncc1)C(=O)N1CCC[C@H]1B(O)O] | −6.60 | 14.07 | 3.657 |
| HYNIC-iFAP 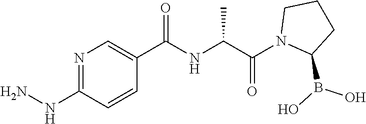 [SMILES: C[C@@H](NC(=O)c1ccc(NN)nc1)C(=O)N1CCC[C@H]1B(O)O] | −8.53 | 0.536 | 3.274 |
| DOTA-Bz-NCS-HYNIC-iFAP 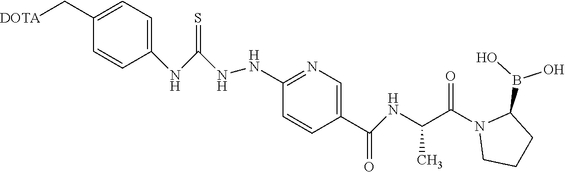 | −8.89 | 0.292 | 4.496 |

Having sufficiently described my invention, I consider as a novelty and therefore claim as my exclusive property, the contents of the following clauses:

1. A radiopharmaceutical of the chemical formula ^{98m}Tc-EDDA/HYNIC-iFAP (^{98m}Tc-HYNIC-iFAP), comprising the structure:

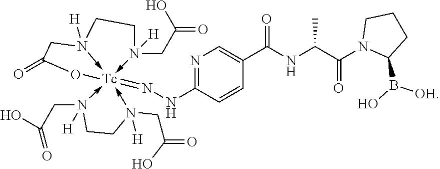

2. A radiopharmaceutical composition, characterized in that it comprises the radiopharmaceutical as claimed in claim 1.

3. The radiopharmaceutical claimed in claim 1 for use as a radiodiagnostic agent.

4. The precursor ligand of radiopharmaceuticals comprising the structure: [chemical structure]
